(19) United States
(12) Patent Application Publication
(10) Pub. No.: US 2026/0216321 A1
(43) Pub. Date: Jul. 30, 2026

(54) HYPOALLERGENIC DERIVATIVES OF THE IMMUNODOMINANT PEANUT ALLERGEN

(71) Applicant: California Northstate University, Elk Grove, CA (US)

(72) Inventors: Catherine Yang, Elk Grove, CA (US); Eslam Mohamed, Elk Grove, CA (US); Ahmed El-Shamy, Elk Grove, CA (US); Alvin Cheung, Sacramento, CA (US)

(21) Appl. No.: 19/197,921

(22) Filed: May 2, 2025

Related U.S. Application Data
(60) Provisional application No. 63/642,010, filed on May 3, 2024.

(51) Int. Cl. A61K 39/35; C07K 14/415

(52) U.S. Cl. CPC A61K 39/35; C07K 14/415

(57) ABSTRACT

The present invention provides compositions and methods for treating peanut allergy. The compositions comprise chemically modified peanut allergen proteins selected from Ara h 1, h 2, h 3, h 6, h 8, and h 9, wherein the proteins are chemically crosslinked using a dialdehyde crosslinking agent and exhibit reduced IgE binding affinity. Methods of treatment comprise administering to a subject a composition containing at least one chemically crosslinked peanut allergen protein selected from Ara h 1, h 2, h 3, h 6, h 8, and h 9. The composition is administered in an amount effective to induce desensitization or immune tolerance to peanut allergens. The chemically modified peanut allergen proteins may be formulated in various dosage forms suitable for oral, subcutaneous, intramuscular or epicutaneous administration. The invention also encompasses compositions wherein the modified peanut allergen proteins are combined with excipients that enhance stability, delivery, or immunomodulatory properties.

### FIELD OF THE INVENTION

This application relates to a novel protein modification to reduce its immune reactivity. Particularly, this applicate relates to the development of allergoids by inducing crosslinking of the monomeric form of a specific peanut allergen.

### BACKGROUND

Peanut allergy is the leading cause of life-threatening and detrimental food allergy in the United States. Unlike some food allergies that often resolve with age, peanut allergy typically persists into adulthood. The childhood prevalence of peanut allergy in the U.S. has notably increased over the last decade, with a range from 1.2% to 2.2% of the population affected. Therefore, as peanut allergy prevails over time, the cost of care and its overall burden over the U.S. economy has been risen significantly.

The first line of managing a peanut allergy is avoiding peanuts and their products in the diet and educating the patient on how to quickly respond to an allergic reaction. However, the impact of avoidance and fear of exposure to the allergen by accident throughout the lifetime can negatively impact on the patient's quality of life due to the wide availability of peanut containing food products in general food space. On Jan. 31, 2020, the U.S. FDA officially approved PALFORZIA™ as a standard of care product for peanut allergy for patients 4 to 17 years of age. PALFORZIA™ is an oral immunotherapy treatment that aims to reprogram the immune system not to react severely against the allergen by raising the threshold of the immune responses triggered during an allergic reaction. This method of intervention is also known as “Peanut desensitization”.

Even with the successful therapeutic outcome of PALFORZIA™, itis not actually considered as a real cure for peanut allergy. There are multiple side effects associated with administration of PALFORZIA™ ranging from oral itching and swelling, abdominal pain, nausea, vomiting, diarrhea, urticaria, rhinitis, and in most severe cases, anaphylaxis. Therefore, patients under treatment still need to carry epinephrine with them all the time. Another way to decrease the side effects of PALFORZIA™ is to combine it with omalizumab (Xolair) which is a monoclonal antibody against IgE. Moreover, PALFORZIA™ is only recommended within 4 to 17 years of age which leaves a wide range of adult patients without potential treatment so far.

Experiments conducted to induce immune tolerance to allergens by administrating modified allergens as chemically altered allergoids, adjuvant-coupled allergen and nanoparticles coated allergens showed promising results that paved the way to develop allergen specific immunotherapy. The rationale for inducing those modifications is to ensure a reduced IgE reactivity with altered 3-D structure of the antigenic determinants of the allergen but retaining T cell differentiation to subsets that promote tolerance instead. However, despite the research going in the field of allergen modification, there is still an urgent need for more modified allergen candidates for clinical studies to evaluate their vaccine potency in patients.

### SUMMARY

The present application provides a composition of peanut allergen protein, comprising at least one peanut allergen protein selected from the group consisting of: Ara h 1, 2, 3, 6, 8, and 9, wherein the peanut allergen protein is chemically modified, and wherein the chemically modified peanut allergen protein demonstrated reduced binding affinity with IgE.

According to one aspect of the invention, the chemical modification is by crosslinking. According to one embodiment of the invention, the chemical modification is by crosslinking with a dialdehyde. More preferably the crosslinking agent can be a diphenyl-aldehyde. In a specific exemplary embodiment, the chemical modification is by crosslinking with a glyoxal, preferably a phenyl glyoxal. Some of the reagents for the chemical modification is at least one selected from phenylglyoxal, 4-methylphenylglyoxal, difluorophenylglyoxal, 3,4-difluorophenylglyoxal, 4-fluoro-phenylglyoxal, 2,4-4-chlorophenylglyoxal, 4-hydroxyphenylglyoxal, 3-carbomethoxy-4-hydroxyphenylglyoxal, 4-morpholino-phenylglyoxal, and 4-nitrophenylglyoxal. Preferred exemplary crosslinking agents are phenylglyoxal or 4-Fluorophenylglyoxal.

According to another aspect of the invention, the chemically modified peanut allergen protein having an average molecular weight greater than an average molecular wight of a native peanut allergen protein. In one embodiment of the invention, the chemically modified peanut allergen protein having an average molecular weight about 1-2 fold of an average molecular wight of a native peanut allergen protein. In another embodiment of the invention, the chemically modified peanut allergen protein having an average molecular weight about 2-fold of an average molecular wight of a native peanut allergen protein.

According to a further aspect of the invention, the chemically modified peanut allergen protein is Ara h 1. Ara h 2, and Ara h 6.

The present invention also contemplates formulating the chemically modified composition of peanut allergen protein with an excipient.

The present invention further contemplates a solid dosage form of the chemically modified composition of peanut allergen protein.

The present invention also contemplates a liquid dosage form of the chemically modified composition of peanut allergen protein.

The present invention provides methods for treating peanut allergy in a subject by administering a composition comprising a chemically crosslinked peanut allergoid. The allergoid includes at least one peanut protein selected from Ara h 1, Ara h 2, Ara h 3, Ara h 6, Ara h 8, and Ara h 9, wherein the peanut protein is chemically crosslinked using a dialdehyde crosslinking agent, such as glyoxal. The composition is administered in an amount effective to induce desensitization or immune tolerance to peanut allergens.

In certain embodiments, the chemically modified peanut allergen protein comprises at least one of Ara h 1, Ara h 2, or Ara h 6. The method may involve administration according to a dose-escalation protocol comprising an initiation phase, a build-up phase, and a maintenance phase. The composition may be administered orally once daily to once weekly, subcutaneously or intramuscularly at intervals of one to four weeks, or epicutaneously. In certain embodiments, the composition may further comprise an adjuvant.

In certain aspects, the method includes initiating treatment with a dose containing less than 5 mg of peanut protein equivalent, with incremental increases to a maintenance dose of at least 300 mg of peanut protein equivalent. The chemically crosslinked peanut allergoid exhibits reduced IgE-binding capacity relative to the corresponding unmodified peanut protein.

### BRIEF DESCRIPTION OF DRAWINGS

The foregoing summary, as well as the following detailed description of the preferred embodiments, will be better understood when read in conjunction with the appended drawings. It should be understood, however, that the invention is not limited to the precise arrangements and instrumentalities shown. In the drawings,

FIG. 1: Schematic diagram illustrating the main idea of the invention and the ultimate therapeutic goal of developing Ara h 2 allergoids;

FIG. 2a: Coomassie colloidal staining of Native Ara h 2 and Ara h 2 allergoids formed by cross-linking reactions with 16.67, 33.33, and 66.67 mM concentrations of phenylglyoxal. (number of experiments performed=3);

FIG. 2b: Western blot of native Ara h 2 and Ara h 2 allergoids formed by cross-linking reactions with three 16.67, 33.33, and 66.67 mM concentrations of phenylglyoxal. The Native Ara h 2 and three phenylglyoxal cross-linked allergoids were probed using a monoclonal human anti-Ara h 2 IgE primary antibody (number of experiments performed=3);

FIG. 2c: Sandwich ELISA detection of native and phenylglyoxal cross-linked Ara h 2 allergoids (PG-Ara h 2), using wells coated with monoclonal human anti-Ara h 2 IgE primary antibody, detection using a polyclonal rabbit anti-Ara h 2 IgG primary antibody and HRP-conjugated anti-rabbit IgG. Error bars represent ranges in O.D. as performed over 3 separate experimental trials. Statistical analysis was performed by two-way ANOVA, n=3 with mean±SEM ***p<0.001;

FIG. 3: Results describing changes in serum antibody binding to native and PG-Ara h 2) when comparing serum samples from healthy (n=2) and patients with peanut allergy (n=3);

FIG. 4: Flow cytometry analysis to test the reactivity of serum-sensitized LAD2 mast cells towards native Ara h 2 and PG-Ara h 2. FIG. (A) represents the experiment layout for sensitizing and challenging LAD2 mast cells, FIGS. (B-C) are histograms representing the surface expression of CD63 (B) and CD107a (C) in LAD2 mast cells. The expression intensity of the tested surface markers is shown by mean fluorescence index (MFI);

FIG. 5a: Coomassie colloidal staining of Native Ara h 2 and Ara h 2 allergoids formed by cross-linking reactions with 16.67, 33.33, and 66.67 mM concentrations of 4-Fluorophenylglyoxal. (number of experiments performed=3);

FIG. 5b: Western blot of native Ara h 2 and Ara h 2 allergoids formed by cross-linking reactions with three 16.67, 33.33, and 66.67 mM concentrations of 4-Fluorophenylglyoxal. The Native Ara h 2 and three 4-Fluorophenylglyoxal cross-linked allergoids probed using a monoclonal human anti-Ara h 2 IgE primary antibody (number of experiments performed=3);

FIG. 5c: Sandwich ELISA detection of native and 4-Flourophenylglyoxal cross-linked Ara h 2 allergoids (4F-PG-Ara h2), using wells coated in monoclonal human anti-Ara h 2 IgE primary antibody, detection using a polyclonal rabbit anti-Ara h 2 IgG primary antibody and HRP-conjugated anti-rabbit IgG. Error bars represent ranges in O.D. as performed over 3 separate experimental trials. Statistical analysis was performed by two-way ANOVA, n=3 with mean±SEM ***p<0.001;

FIG. 6: Results describing changes in serum antibody binding to native and 4F-PG-Ara h2 when comparing serum samples from healthy (n=2) and patients with peanut allergy (n=3);

FIG. 7: Flow cytometry analysis to test the reactivity of serum sensitized LAD2 mast cells towards native Ara h 2 and 4F-PG-Ara h 2. FIG. (A) represents the experiment layout for sensitizing and challenging LAD2 mast cells, FIGS. (B-C) are histograms representing the surface expression of CD63 (B) and CD107a (C) in LAD2 mast cells. The expression intensity of the tested surface markers is shown by mean fluorescence index (MFI); and

FIGS. 8A, 8B, and 8C: Results of evaluating PG-Ara h 2 and 4F-PG-Ara h 2 allergoids in peanut sensitized C3H/HeJ mice. FIG. 8A shows clinical symptom scores, FIG. 8B shows the mean body temperatures, and FIG. 8C indicates serum histamine concentrations in mice following challenge with PBS, Ara h 2, PG-Ara h 2, or 4F-PG-Ara h 2 (n=4 per group). Data are expressed as the mean±SEM. Statistical significance was evaluated using Student's t-test (P<0.01).

### DETAILED DESCRIPTION

Peanut allergy is a prevalent IgE-mediated food allergy known for its potential to induce severe anaphylactic reactions, a life-threatening condition. There are over 13 allergenic components in peanuts, with key markers including Ara h 1, 2, 3, 6, 8, and 9. Among these, Ara h 1, 2, and 3, categorized as seed storage proteins, pose a high risk of systemic allergic reactions, particularly anaphylaxis. Ara h 2 is a more important predictor of clinical peanut allergy than Ara h 1 and 3, and is most often associated with severe allergic reactions.

Ara h 2 is a 2S albumin storage protein. The structure of Ara h 2 is characterized by a distinctive five-helix bundle arrangement, secured by four disulfide bonds, thus conferring stability to its overall conformation. Ara h 2 is relatively small with a molecular weight ranging approximately between 17 to 19 kilodaltons (kDa). It is heat-stable and resistant to digestion within the gastrointestinal tract.

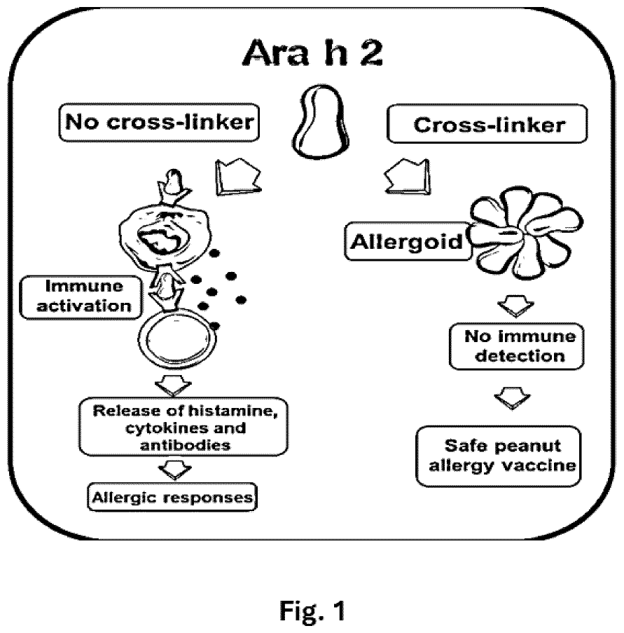

The present application provides a method of developing new hypoallergenic variants (allergoids) aiming to utilize those derivatives in a safe allergen-specific immunotherapy in patients with peanut allergy (FIG. 1). In the particular example of Ara h 2, a crosslinking agent is used modify the peanut allergen protein, whereby weakening or disrupting reactivity with IgE, and also may produce oligomerized peanut allergen protein, preferably dimer of Ara h 2, which can be used for the purpose of immune desensitization treatment of peanut allergy.

In a first embodiment, cross-linker Phenylglyoxal (PG), is used to generate an allergoid product, herein referred to as PV-1015. The allergoids were created after optimizing various conditions including incubation time, allergen concentration, crosslinker concentration, reaction volume, reaction temperature and other incubation settings. A particular example is described below.

### Confirmation of Crosslinking Reaction by Coomassie Colloidal Staining

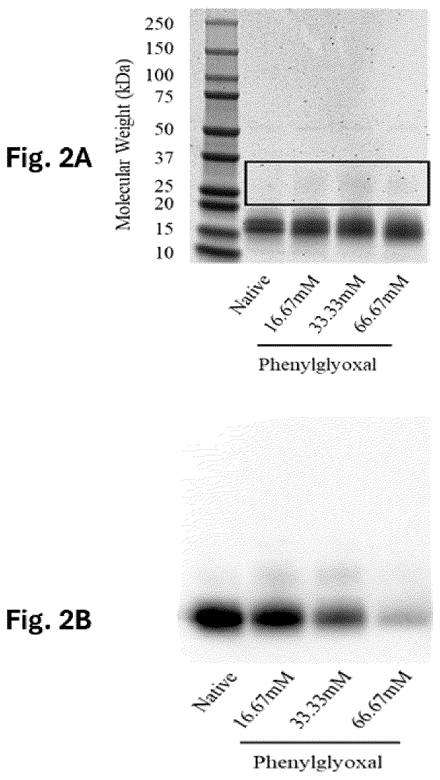

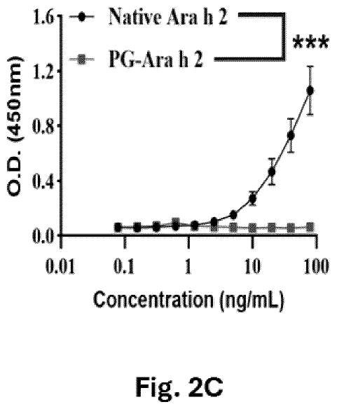

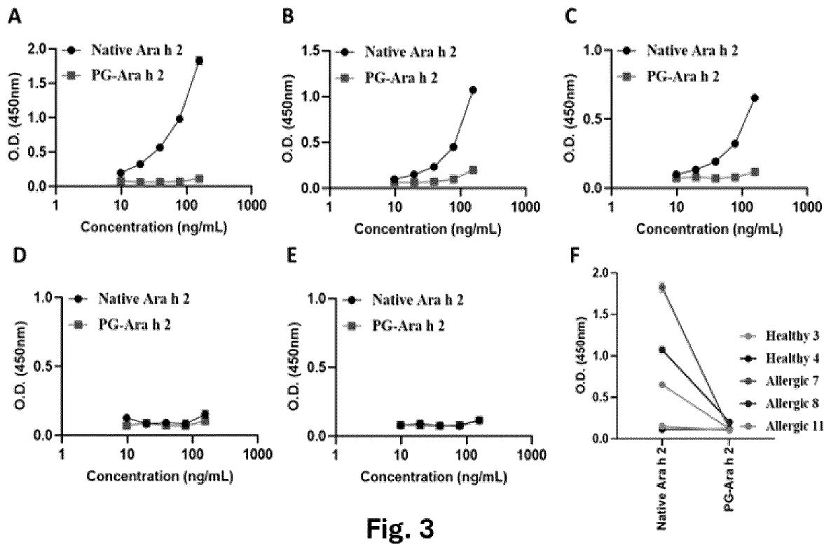

One approach to test the formation of the dimerized allergoid, PV-1015 following the reaction of native Ara h 2 with PG was to perform SDS PAGE with Coomassie Blue staining to visualize the protein bands according to methods illustrated in Examples 1a, 1b, and 1c. The cross-linking reaction was performed by incubating 2 ug of Ara h 2 with three different concentrations (16.67 mM. 33.33 mM, and 66.67 mM) of PG in HEPES buffer for 24 hours at room temperature (FIG. 2A). The untreated Ara h 2 (Native) migrates at 17 and 19 kDa forming a doublet band representing the two isoforms of Ara h 2, Ara h 2.0101 and Ara h 2.0201, respectively. It is found that by increasing the concentration of PG in the reaction, the two isoforms doublet band slightly decreased on the gel with the formation of a new band at about 32 kDa, suggesting the formation of a dimerized derivative of Ara h 2.

### Testing the IgE Binding Affinity to PV-1015 in its Linear Format

After confirming the physical properties of PV-1015 through Coomassie staining, we sought to test the immunological properties of this new derivative. To achieve that, we utilized a monoclonal IgE antibody sourced from patients with peanut allergy and performed a western blot to detect the binding of IgE to its epitope as detailed in Example 1d. After the cross-linking reaction, the native Ara h 2 and PV-1015 were electrophoresed, blotted to PVDF membrane, and probed with monoclonal IgE. We found a significant reduction in IgE binding to PV-1015 at the highest concentration of PG (66.67 mM) and to a lesser extent at 33.33 mM PG while no effect was observed at the lowest PG concentration (16.67 mM) or the native Ara h 2 (FIG. 2B). Therefore, PV-1015 exhibits an altered linear epitope determinant with lower affinity to IgE binding compared to its native form.

### Testing the IgE Binding of PV-1015 in its Conformational Format

The western blot technique involves a reduction/denaturation step that disrupts the epitope binding surface of the tertiary structure of proteins mediated by hydrogen-bonding interactions. Despite the interesting observation of disrupted IgE binding to PV-1015 found by western blot, the binding affinity of IgE to PV-1015 in its original three-dimensional structure remains pivotal to be tested. We performed sandwich ELISA through which we assessed the binding of a titrated native Ara h 2 and PV-1015 to a plate bound human monoclonal IgE antibody as described in Example 1e. In this experiment, we tested Ara h 2 cross-linked with 66.67 mM of PG since it was the most potent condition to disrupt IgE binding in the western blot assay. The ELISA assay strikingly revealed that PV-1015 shows an abolished binding to IgE compared to its native counterpart suggesting that the conformational epitope determinant in PV-1015 that binds to IgE is completely masked due to the reaction with PG (FIG. 2C).

Evaluating the Recognition of PV-1015 by Serum IgE from Peanut-Sensitive Patients

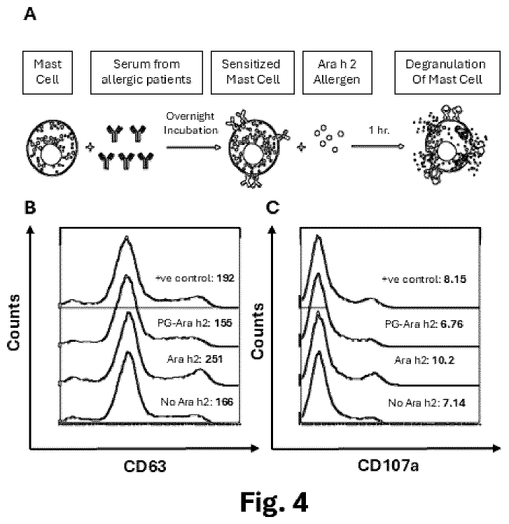

To test the ability of PG to mask the conformational epitopes to polyclonal anti-Ara h 2 IgE, we challenged PV-1015 with sera from patients with peanut allergy to assess the binding of the polyclonal pool of IgE antibodies in those sera to their corresponding epitopes in both native and Ara h 2 derivate. We also used sera from healthy individuals and tested them against our targets to confirm the anti-native Ara h 2 signal that we obtained from patient's sera. We performed a direct ELISA assay in which serum samples were incubated with a plate bound and titrated Ara h 2 (Native and PV-1015) according to the method shown in Example 1f. FIG. 3 shows direct ELISA experiments testing the lgE binding capacity of native Ara h 2 and PG-Ara h 2 (PV-1015) in serum samples collected from peanut sensitive patients and healthy individuals. FIGS. (3A-3C) represent ELISA titration curves from individual patients (n=3), FIGS. (3D-3E) represent healthy donors (n=2) and FIG. (3F) is a collective data figure from all serum samples illustrating the change in optical density at 156.25 ng/ml of native Ara h 2 and PG-Ara h 2. In alignment with the above-described data, PV-1015 wasn't recognized by sera from different patients. On the other hand, the native form of Ara h 2 was detected in a dose response manner by sera from patients but not from healthy individuals.

### Testing the Reactivity of Sensitized Mast Cells to PV-1015

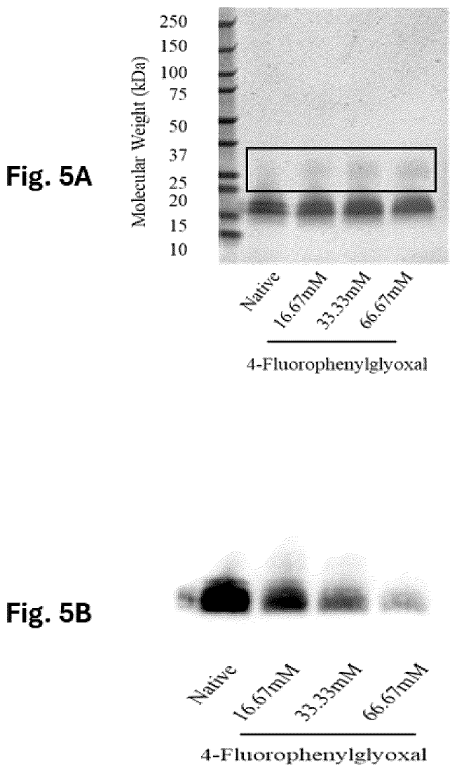

In order to assess the reactivity of mast cells towards PV-1015, we developed an in vitro assay (according to Example 1g) to measure the degranulation capacity of-serum-sensitized LAD2 mast cells following challenging with native Ara h 2 versus its cross-linked format, PV-1015 (FIG. 4A). We found that LAD2 cells showed a heightened degranulation reflected by an increase in their surface expression of CD63 and CD107a (degranulation markers) after their exposure to native Ara h 2 (FIGS. 4B and 4C) Interestingly, LAD2 cells had a lower expression of the degranulation markers in response to PV-1015 suggesting a decrease in reactivity towards the cross-linked allergoid compared to the native form of Ara h 2. To ensure that LAD2 cells are successfully coated with IgE during the sensitization step, we stimulated them with Anti-IgE polyclonal antibody (+ve control) and found that LAD2 cells degranulated accordingly.

In this particular embodiment, we found that the binding capacity of IgE to both linear and conformational epitopes is completely disrupted in PV-1015 compared to the untreated Ara h 2 (Native). Moreover, we confirmed the hypoallergenic nature of our novel compound, PV-1015 in two clinically relevant models. First, we tested its detection by serum samples collected from peanut allergy patients and found that PV-1015 is completely masked from being recognized by the polyclonal IgE pool in serum samples. Then, we showed a lack of reactivity of PV-1015 towards serum-sensitized mast cells that are considered a subset of immune cells involved in allergy-associated inflammatory reactions.

In a second embodiment, cross-linker 4-Fluorophenglyoxal (4F-PG), is used to generate an allergoid product, herein referred to as PV-1016. The allergoids were developed after optimizing various conditions including incubation time, allergen concentration, crosslinker concentration, reaction volume, reaction temperature and other incubation settings. A particular example is described below.

### Confirmation of Crosslinking Reaction by Coomassie Colloidal Staining

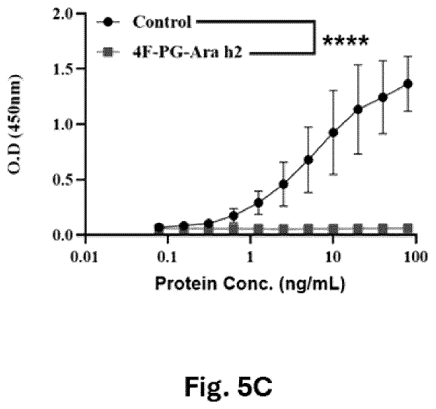

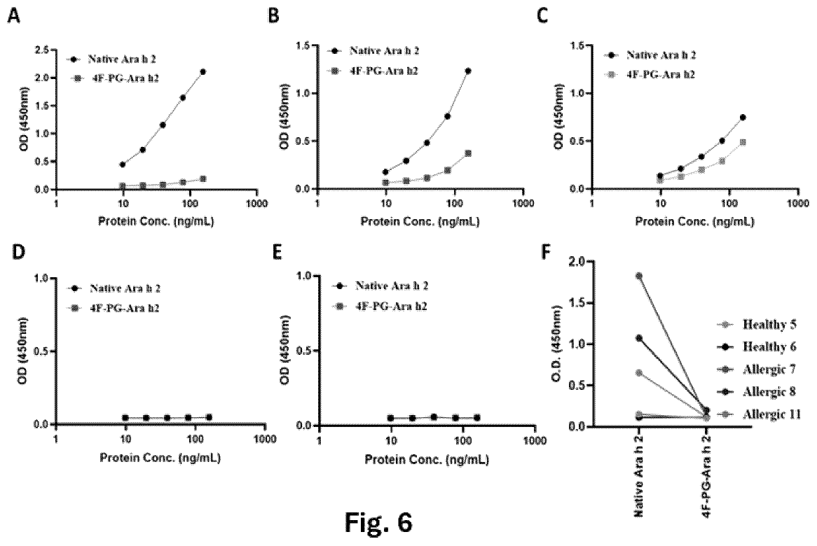

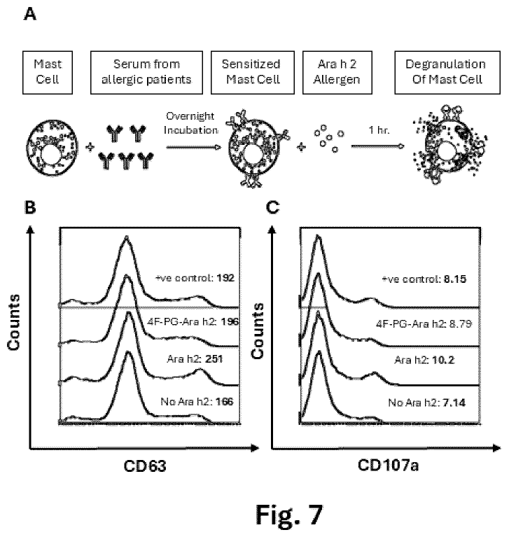

Our first approach to test the formation of the dimerized allergoid, PV-1016 following the reaction of native Ara h 2 with 4F-PG was to perform SDS PAGE with Coomassie Blue staining to visualize the protein bands according to Example 1a, 1b, and 1c. The cross-linking reaction was performed by incubating 2 μg of Ara h 2 with three different concentrations of 4F-PG in HEPES buffer for 24 hours at room temperature (FIG. 5A). The untreated Ara h 2 (Native) migrates at 17 and 19 kDa forming a doublet band representing the two isoforms of Ara h 2, Ara h 2.0101 and Ara h 2.0201, respectively. We found that by increasing the concentration of 4F-PG in the reaction, the two isoforms doublet band slightly decreased on the gel with the formation of a new band at about 32 kDa suggesting the formation of a dimerized derivative of Ara h 2.

### Testing the IgE Binding Affinity to PV-1016 in its Linear Format

After confirming the physical properties of PV-1016 through Coomassie staining, we sought to test the immunological properties of this new derivative. To achieve that, we utilized a monoclonal IgE antibody sourced from patients with peanut allergy and performed a western blot to detect the binding of IgE to its epitope as described in Example 1d. After the cross-linking reaction, the native Ara h2 and PV-1016 were electrophoresed, blotted to PVDF membrane, and probed with monoclonal IgE (FIG. 5B). We found a significant reduction in IgE binding to PV-1016 at the highest concentration of 4F-PG (66.67 mM) and to a lesser extent at 33.33 mM 4F-PG while no effect was observed at the lowest concentration (16.67 mM) or the native Ara h 2. Therefore, PV-1016 exhibits an altered linear epitope determinant with lower affinity to IgE binding compared to its native form.

### Testing the IgE Binding of PV-1016 in its Conformational Format

The western blot technique involves a reduction/denaturation step that disrupts the tertiary structure of proteins mediated by hydrogen-bonding interactions. Despite the interesting observation of disrupted IgE binding to PV-1016 found by western blot, the binding affinity of IgE to PV-1016 in its original three-dimensional structure remains to be tested. We performed sandwich ELISA through which we assessed the binding of a titrated native Ara h 2 and PV-1016 to a plate bound human monoclonal IgE antibody according to the method described in Example 1e. In this experiment, we only tested Ara h 2 cross-linked with 66.67 mM of 4F-PG since it was the most potent condition to disrupt IgE binding in the western blot assay. The ELISA assay strikingly revealed that PV-1016 shows an abolished binding to IgE compared to its native counterpart suggesting that the conformational epitope determinant in PV-1016 that binds to IgE is completely masked due to the reaction with 4F-PG (FIG. 5C).

Evaluating the Recognition of PV-1016 by Serum IgE from Peanut-Sensitive Patients

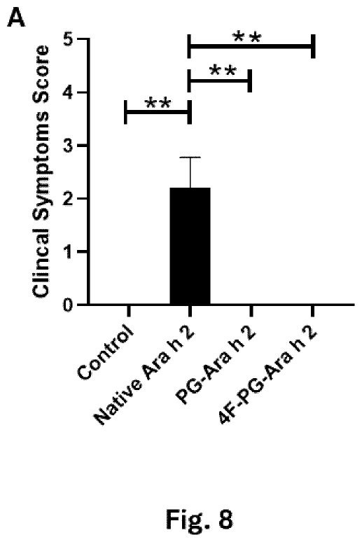

To test the ability of 4F-PG to mask the conformational epitopes of Ara h 2 to polyclonal anti-Ara h 2 IgE, we challenged PV-1016 with sera from patients with peanut allergy to assess the binding of the polyclonal pool of IgE antibodies in those sera to their corresponding epitopes in both native and Ara h 2 derivate. We also used sera from healthy individuals and tested them against our targets to confirm the anti-native Ara h2 signal that we obtained from patient's sera. We performed a direct ELISA assay in which serum samples were incubated with a plate bound with titrated Ara h 2 (Native and PV-1016) according to the method shown in Example 1f. Results are shown in FIG. 6. FIGS. (6A-6C) represent ELISA titration curves from individual patients (n=3), FIGS. (6D-6E) represent healthy donors (n=2), and FIG. (6F) is a collective data figure from all serum samples illustrating the change in optical density at 156.25 ng/ml of native Ara h 2 and 4F-PG-Ara h 2. In alignment with the above-described data, PV-1016 wasn't recognized by sera from different patients. On the other hand, the native form of Ara h 2 was detected in a dose response manner by sera from patients but not from healthy individuals.

### Testing the Reactivity of Sensitized Mast Cells to PV-1016

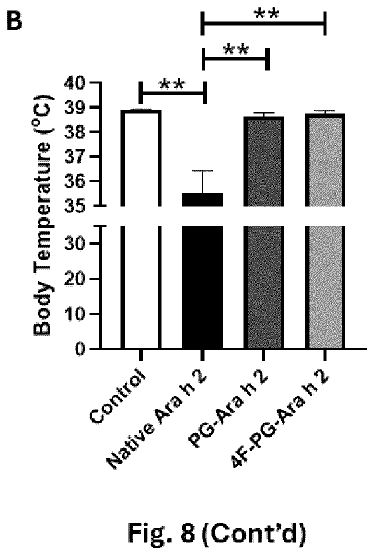

In order to assess the reactivity of mast cells towards PV-1016, we developed an in vitro assay (described in methods section) to measure the degranulation capacity of serum-sensitized LAD2 mast cells following challenging with native Ara h 2 versus its cross-linked format, PV-1016 (FIG. 7A). We found that LAD2 cells showed a heightened degranulation reflected by an increase in their surface expression of CD63 and CD107a (degranulation markers) after their exposure to native Ara h 2. Interestingly, LAD2 cells had a lower expression of the degranulation markers in response to PV-1016 suggesting a decrease in reactivity towards the cross-linked allergoid compared to the native form of Ara h 2 (FIGS. 7B and 7C). To ensure that LAD2 cells are successfully coated with IgE during the sensitization step, we stimulated them with Anti-IgE polyclonal antibody (+ve control) and found that LAD2 cells degranulated accordingly.

We found that the binding capacity of IgE to both linear and conformational epitopes is completely disrupted in PV-1016 compared to the untreated Ara h 2 (Native). Moreover, we confirmed the hypoallergenic nature of our novel compound, PV-1016 in a two clinically relevant models by testing its detection in by specific IgE in serum samples collected from peanut allergy patients and its reactivity towards serum-sensitized mast cells. In both approaches, we found that PV-1016 is completely masked from being recognized by the polyclonal IgE pool in serum samples and failed to induce mast cells degranulation.

Allergoids are Hypoallergenic in Mice with Peanut Allergy

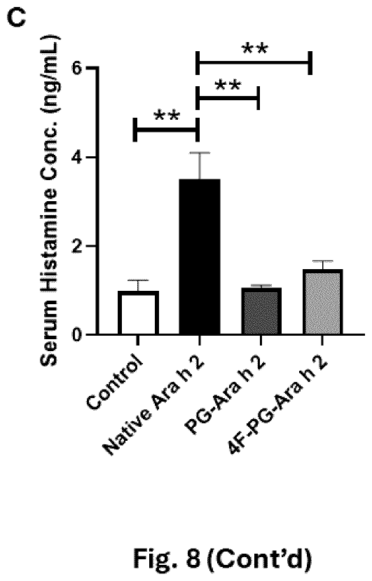

We found that PV-1015 and PV-1016 allergoids are hypoallergenic in mice sensitized by peanut extract (PE) and are well tolerated. Female CH3/HeJ mice were sensitized and challenged according to Example 2. Results are shown in FIGS. 8A-8C. Statistical significance was evaluated using Student's t-test (P<0.01).

Clinical symptoms were observed exclusively in the group of mice challenged with native Ara h 2, with an average clinical score of 2. In contrast, mice administered crosslinked allergoids exhibited no clinical symptoms, comparable to the vehicle (control) group (FIG. 8A).

To further assess the physiological response to allergen exposure, rectal body temperature was monitored. A significant decrease in body temperature was detected in mice challenged with native Ara h 2, while no notable changes were observed in the crosslinked or control groups. This temperature reduction is indicative of hypothermia, a known marker of systemic anaphylaxis (FIG. 8B).

Additionally, serum histamine levels were measured, revealing a significant elevation in the native Ara h 2 group relative to both the crosslinked and control groups. This finding suggests that peanut sensitized mice exposed to native Ara h 2 experienced an immediate hypersensitivity reaction. Collectively, our results demonstrate that PV-1015 and PV-1016 exhibit reduced allergenicity in a murine model of peanut allergy (FIG. 8C).

### Allergoids can be Used to Desensitize Mice Sensitized by Peanut Extract

Female C3H/HeJ mice (4 weeks old) will be sensitized with PE following the protocol described in example 2. After 6 weeks of sensitization, mice will undergo desensitization via intraperitoneal injections administered once weekly for four weeks. Treatment groups will include vehicle, 100 μg of PG-Ara h 2, or 100 μg of 4F-PG-Ara h 2. At the end of the desensitization period, mice will be challenged intraperitoneally with either vehicle or 100 μg of native Ara h 2. Rectal temperature will be recorded at 5-minute intervals for 15 minutes post-challenge to monitor for hypothermia. Clinical symptoms will be assessed and scored using a standardized clinical scoring system. Finally, blood samples will be collected, and serum histamine levels will be quantified using a commercial ELISA kit. We expect PG and 4F-PG allergoids to be effective at desensitizing mice previously sensitized by peanut extract.

### EXAMPLES

1a. Chemical Crosslinking:

Purified Ara h 2 was obtained from Indoor Biotechnologies (Product Code: NA-AH2-1) at an initial concentration of 1.5 mg/ml in 1×PBS. Phenylglyoxal monohydrate (PG) (Sigma-Aldrich, CAS Number 1075-06-5) was prepared at varying concentrations in HEPES buffer with 10% pure methanol added and adjusted pH at 7.5. 4-Fluorophenylglyoxal hydrate (4F-PG) (Alfa Aesar, CAS: 403-32-7) was prepared at varying concentrations in HEPES buffer with 10% pure methanol and 10% ethanol adjusted pH at 7.5. Varying concentrations of PG and 4F-PG were tested with 2 μg of Ara h 2 protein. The reactions were carried out in 0.2 ml PCR Tubes (Genesee Scientific, Cat. No. 22-154) in an enclosed orbital shaker (25° C., 60 RPM) for 24 hours in the absence of light.

1b. SDS PAGE:

Protein samples were prepared for gel electrophoresis in Laemmli Buffer (BioRad 2× Laemmli Sample Buffer, Cat. #1610737). The samples were all heated to 90-95° C. on a dry heat block for 5 minutes. Gel electrophoresis was performed using pre-cast protein gels (BioRad 4-20% Protein Gels, Cat. #4561093) at 100V in 1× Tris-Glycine Running Buffer containing 0.1% SDS (diluted from 10× stock, BioRad 10× Tris/Glycine Buffer Cat. #1610734). A standard molecular ladder was used (BioRad Precision Plus Protein Dual Color Standards, Cat. #1610374).

1c. Coomassie Staining:

Gels were stained in a colloidal Coomassie dye G-250 (ThermoFisher Scientific GelCode™ Blue Stain Regeant, Cat. No. 24592) overnight at 4° C. The gels were then washed in Milli-Q water until the excess stain was removed. The gels were imaged at 700 nm and 800 nm, and the images were obtained (LI-COR Odyssey CLx Imaging System).

1d. Western Blot:

After electrophoresis, the gels were rinsed in Milli-Q water and equilibrated in transfer buffer (1× Tris-Glycine, 20% Methanol). The proteins were transferred onto PVDF membranes (BioRad Immuno-Blot PVDF Membrane, Cat. #1620177) overnight at 4° C., 35V. Following transfer, the membranes were imaged with Ponceau to assess transfer efficacy. After washing the Ponceau off with TBS-T (1×TBS, 0.1% Tween, Sigma-Aldrich Cat. #9005-64-5), the membranes were blocked in 5% non-fat dry milk (NFDM) for 2 hours at room temperature. Membranes were then incubated overnight in primary antibody (monoclonal anti-Ara h 2 human IgE from an allergic patient (Indoor Biotechnologies, Product Code: E-16A8)) at a ratio of 1:1000 in 5% nonfat dry milk at 4° C. The primary antibody was washed off, and the membrane was washed in TBS-T. The membrane was then stained incubated in secondary antibody (HRP-conjugated mouse anti-human IgE antibody (Invitrogen REF SA5-10306)) for 2 hours. The proteins were then imaged using ECL substrate (ThermoScientific Cat. No. 32106) and an appropriate imager (Azure Biosystems c600 Imager).

1e. Sandwich ELISA Using IgE Monoclonal Antibody from Allergic Patients.

Briefly, 96-well ELISA plates (Nunc MaxiSorp™ ELISA plates uncoated Biorad, Cat #423501) were coated overnight at 4° C. in monoclonal anti-Ara h 2 IgE capture antibody (Indoor Biotechnologies, Product Code: E-16A8) diluted at a ratio of 1:1000 in coating buffer (carbonate-bicarbonate buffer, pH 9.5). The coating buffer was washed off with PBS containing 0.05% Tween (PBS-T, ThermoFisher Scientific Product No. 28372 and Tween 20), and the plates were blocked with 1% BSA for 1 hour at room temperature. This was followed by the addition of increasing concentrations of Ara h 2 protein (either crosslinked or non-crosslinked, diluted in 1% BSA, and desalted via 7K MWCO column, ThermoFisher Scientific Cat. No. 89877). The plates were then incubated for 1 hour at room temperature. After washing off the protein samples, the plates were then incubated with a primary detection antibody specific to Ara h 2 (anti-Ara h 2 polyclonal rabbit IgG, InBio Product Code: PA-AH2) diluted in 5% BSA. for 1 hour at room temperature. After washing off the primary detection antibody, the plates were then incubated with a secondary antibody (HRP-conjugated anti-rabbit IgG, Cell Signaling 7074P2) for 1 hour at room temperature. After washing off the secondary antibody, antibody binding was measured using TMB substrate (BioLegend Cat. No. 421101), followed by stop solution (BioLegend Cat. No. 423001) and reading O.D. at 450 nm (Thermo VarioSkan Flash plate reader).

1f. Direct ELISA Using Allergic Patient Sera.

Briefly, 96-well ELISA plates were coated overnight at 4° C. with crosslinked and non-crosslinked Ara h 2 protein samples titrated in coating buffer. The plate was then washed with PBS-T, and blocked with 1% BSA. The coated proteins were then incubated with either peanut-allergic or healthy human serum diluted 1:10 in blocking buffer (samples are sourced from BioIVT under IRB approved protocol). The serum was washed off and protein-antibody binding was detected by HRP-conjugated mouse anti-human IgE secondary antibody. The activity of HRP was measured using TMB substrate as above after washing off the secondary antibody.

1g. Evaluating Mast Cell Activation in Response to Allergoids

LAD2 mast cells were purchased from abm Inc. Cat #T8157. Cells were incubated with 100 ng/ml IL-4 for 5 days then sensitized overnight with serum from patients with peanut allergy to ensure cells can detect the Ara h2 protein prior to the challenge assay. To induce the allergic response, sensitized LAD2 cells were incubated with 2 ug of crosslinked Ara h2 or native Ara h2 for 10 minutes. As a positive control, we challenged the LAD2 cells with polyclonal anti-IgE to confirm LAD2 cells were coated with IgE found in sera of allergic patients. After the 10 minutes of incubation time, cells were collected and stained with CD63 (BioLegend CAS:353008) and CD107a (BD Pharmagen, CAS:555801) to assess their degranulation in response to challenging with native and crosslinked Ara h2. Data was acquired by flow Cytometry using BD FACSCalibur flow cytometer and the was analyzed with FlowJo software (FlowJo 10.7.2).

### 2. Evaluating Mice Anaphylaxis Reaction in Response to Allergoids

Female CH3/HeJ mice (4 weeks old) were obtained from The Jackson Laboratory (Strain #: 000659). Epicutaneous sensitization was performed by applying 5 mg of crude peanut extract (PE) onto the shaved abdominal skin using a cotton swab. Sensitization was conducted twice weekly for a total duration of six weeks. Upon completion, mice were randomly assigned to four experimental groups for intraperitoneal challenge: Group 1 received vehicle (control), Group 2 received 100 μg of native Ara h 2, Group 3 received 100 μg of Ara h 2 crosslinked with phenylglyoxal (PG-Ara h 2), and Group 4 received 100 μg of Ara h 2 crosslinked with 4-fluorophenylglyoxal (4F-PG-Ara h 2). Post-challenge, body temperatures were recorded using rectal thermometer over a 10-minute period to monitor for hypothermia indicative of an anaphylactic response. Clinical symptoms were evaluated using a standardized scoring system: 0=no symptoms; 1=scratching and rubbing around the snout and head; 2=puffiness around the eyes and snout, reduced activity, and increased respiratory rate; 3=wheezing and labored respiration; 4=immobility following prodding or presence of tremors/convulsions; 5=death. Following euthanasia, blood samples were collected, and plasma histamine levels were quantified using an ELISA-based histamine detection kit.

Crosslinkers, also known as bifunctional crosslinkers, are reagents that contain two or more reactive groups which covalently attach via a spacer to functional groups on proteins or other biomolecules. Three types of crosslinkers are available: homobifunctional crosslinkers, heterobifunctional crosslinkers, and photoreactive crosslinkers. Homobifunctional crosslinking reagents have identical reactive groups, primarily amine-to-amine or sulfhydryl-to-sulfhydryl. They are typically used to form intramolecular crosslinks or to prepare polymers from monomers. Heterobifunctional crosslinking reagents have different reactive groups such as amine-to-sulfhydryl, carboxyl-to-amine, or sulfhydryl-to-carboxyl. They are useful for preparing conjugates between two different biomolecules. Heterobifunctional crosslinking reagents also include photoreactive crosslinking reagents that react with nucleophiles or form C-H insertion sites after exposure to UV light.

Cleavable cross-linkers, susceptible to specific conditions like reducing agents, can be cleaved to release the cross-linked products. Conversely, uncleavable cross-linkers form irreversible bonds and resist cleavage under physiological conditions.

Cross-linkers with short spacer arms bring linked molecules into close proximity, whereas those with longer spacer arms offer flexibility, reducing steric hindrance between the linked molecules.

Oligomerized proteins, can be used as allergoids for the purpose of immune-desensitization, particularly within allergen-specific immunotherapy (AIT). These allergoids are derived from allergenic proteins and undergo chemical modification to reduce their allergenicity while retaining their immunogenic properties. The process of oligomerization involves crosslinking allergenic proteins using agents as glutaraldehyde, formaldehyde, phenylglyoxal, or 4-fluorophenylglyoxal, resulting in the formation of stable complexes or oligomers.

Oligomerization in allergoid development reduces the allergenicity of the proteins by altering their structures, thus diminishing their ability to trigger allergic reactions. Despite this reduction in allergenicity, allergoids maintain their immunogenicity, allowing them to induce immune responses and promote immunological tolerance effectively. Additionally, the enhanced stability achieved through oligomerization extends the duration of action of allergoids in the body, thereby improving their efficacy.

Immuno-desensitization with oligomerized allergoids has shown promising results in alleviating allergic symptoms associated with conditions such as allergic rhinitis, asthma, and insect venom allergies. Furthermore, allergoids offer a safer alternative to traditional allergen extracts, reducing the risk of severe allergic reactions during treatment.

The chemically modified peanut allergen protein composition is administered through various routes of administration, including oral ingestion, subcutaneous injection, or intramuscular injection. The dosage form containing the modified peanut allergen protein can be configured to release the protein at a specific rate using controlled-release formulations. However, challenges associated with administering the chemically modified peanut allergen protein through these routes may include poor bioavailability, immunogenicity, and potential for anaphylaxis. These challenges can be addressed through careful formulation and administration of the dosage form, as well as monitoring patients for adverse reactions.

The specific allergen used in the peanut allergen protein composition may vary depending on factors such as patient history and sensitivities. Other types of allergens that could be used in a similar composition include milk allergen protein (casein), egg white allergen protein, soybean allergen protein, and tree nut allergen protein. The choice of crosslinking agent can affect the properties of the resulting modified proteins, such as their stability, efficacy, and immunogenicity.

Chemical modification by crosslinking is an aspect of the present disclosure, which involves modifying at least one peanut allergen protein selected from Ara h 1, 2, 3, 6, 8, and 9 to reduce its binding affinity with IgE. The modified peanut allergen protein has undergone chemical modification by crosslinking using dialdehyde, glyoxal, phenylglyoxal or 4-Fluorophenylglyoxal, as well as other suitable reagents. The use of crosslinking agents results in the formation of covalent bonds between adjacent amino acids in the allergen protein, leading to changes in its conformation and structure. This modification can result in an average molecular weight greater than or equal to that of a native peanut allergen protein, which may improve the efficacy and safety of the modified protein for use in desensitization therapy.

Crosslinking agents used for this purpose include dialdehyde, glyoxal, phenylglyoxal, 4-Fluorophenylglyoxal among others listed in Table 1. The choice of crosslinking agent with added electron-withdrawing- and electron-donating groups on the benzene ring of the parent phenylglyoxal compound can affect the properties of the resulting modified proteins, such as their stability, efficacy, and immunogenicity.

Crosslinking with Dialdehyde

Dialdehyde works by forming covalent bonds between arginine and/or lysine residues in the protein, leading to increased stability and reduced binding affinity with IgE. This modification can result in an average molecular weight greater than or equal to that of a native peanut allergen protein, making it suitable for use in desensitization therapy. The optimal conditions for crosslinking using dialdehyde may be carefully optimized to achieve the desired degree of modification.

Phenylglyoxal and 4-fluorophenylglyoxal are compounds belonging to the glyoxal family, known for their ability to react with primary amines in proteins and biomolecules, facilitating protein modification and crosslinking.

Phenylglyoxal, with the molecular formula C₈H₆O₂, features a phenyl group (C6H5) attached to a glyoxal moiety. It serves as a valuable reagent for detecting and quantifying primary amines in proteins. Through specific reactions with primary amines, phenylglyoxal forms stable adducts, enabling precise labeling and identification of proteins and peptides within biological samples.

Similarly, 4-fluorophenylglyoxal, with the molecular formula C₈H₅FO₂, possesses a structure akin to phenylglyoxal but includes a fluorine atom (F) attached to the phenyl ring at the 4-position. This fluorine substitution enhances the compound's nucleophilic addition capability, making it particularly useful for protein modification and crosslinking studies. Researchers leverage its unique reactivity to explore intricate interactions and modifications within proteins and biomolecules, advancing our understanding of complex biological processes.

Phenylglyoxal notably targets arginine or lysine residues in proteins, facilitating protein modification and crosslinking studies. Similarly, 4-fluorophenylglyoxal exhibits enhanced reactivity towards arginine residues, facilitating protein modification and crosslinking studies with high specificity.

Additional phenylglyoxal compounds that are suitable for the modification of the peanut allergen proteins are listed in Table 1.

TABLE 1
Names, abbreviations and structural formulas of phenylglyoxal

| # | Name | Abbreviation | Chemical Structure |
|---|---|---|---|
| 1 | Phenylglyoxal | PG | 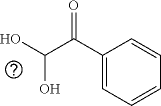 [SMILES: O=C(c1ccccc1)C(O)O] |
| 2 | 4-methylphenylglyoxal | Me-PG | 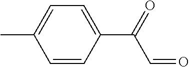 |
| 3 | 4-methoxyphenylglyoxal | MeO-PG | 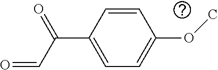 |
| 4 | 4-fluorophenyglyoxal | 4F-PG | 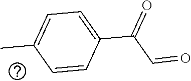 |
| 5 | 2,4-difluorophenylglyoxal | 2,4-diF-PG | 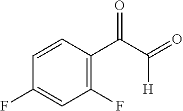 [SMILES: O=CC(=O)c1ccc(F)cc1F] |
| 6 | 3,4-difluorophenylglyoxal | 3,4-diF-PG | 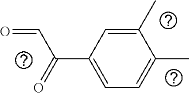 |
| 7 | 4-chlorophenylglyoxal | Cl-PG | 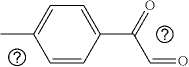 |
| 8 | 4-hydroxyphenylglyoxal | OH-PG | 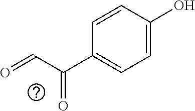 [SMILES: O=CC(=O)c1ccc(O)cc1] |
| 9 | 3-carbomethoxy-4- hydroxyphenylglyoxal | Cam [symbol] OH-PG | 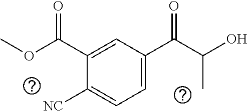 [SMILES: COC(=O)c1cc(C(=O)C(C)O)ccc1C#N] |
| 10 | 4-morpholinophenylglyoxal | Mor-PG | 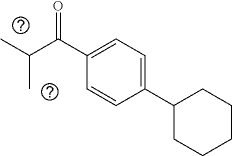 [SMILES: CC(C)C(=O)c1ccc(C2CCCCC2)cc1] |
| 11 | 4-nitrophenyglyoxal | NO-PG | 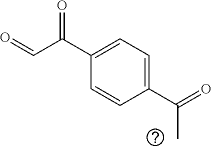 |

[symbol]

indicates data missing or illegible when filed

Modification and crosslinking of proteins using compounds as phenylglyoxal and 4-fluorophenylglyoxal, can also create oligomers of the crosslinked proteins. Oligomers may range from dimers, consisting of two crosslinked protein subunits, to larger complexes comprising multiple interconnected subunits.

The molecular weight of the modified Ara h 2 protein can have an impact on its efficacy and safety for use in desensitization therapy. Higher molecular weights indicate greater level of oligomerization and lower IgE binding activity. When the average molecular weight of the modified protein is about 2-fold of the native (unmodified) protein, it indicates that the modified protein is predominantly in dimer form. A higher average molecular weight would indicate higher order of oligomerization.

The use of chemically modified Ara h 2 in desensitization therapy can potentially reduce the risk of anaphylaxis during administration, although potential side effects such as local reactions at the site of injection, allergic reactions to the modified protein itself, and increased risk of anaphylaxis may still occur. These risks can be minimized through careful monitoring of patients and adjustment of dosages as needed.

In addition to Ara h 2, other Ara h allergens that could be used in a similar composition include Ara h 1, 3, 6, 8, and 9. Ara h1 is another major allergen protein present in peanuts, comprising 12-16% of the total protein in peanut extracts. It belongs to the 7S globulin family, it functions as a vicilin seed storage protein with a molecular weight of approximately 65 kilodaltons (kDa), Ara h1 is heat-stable and resistant to the gastrointestinal tract's harsh conditions.

Ara h 3, is a cupin allergen belonging to the legumin family. Legumins, or 11S globulins, are hexameric proteins found in various plant seeds. Each subunit is initially synthesized as a single polypeptide, later cleaved into acidic and basic polypeptide chains linked by a disulfide bond. Ara h 3 has a molecular weight of 60 kilodaltons (kDa). Cupins, including Ara h 3, exhibit significant resistance to heat treatment and enzymatic activity. There are two isoforms of Ara h 3 identified: Ara h 3.0101 and Ara h 3.0201. These isoforms may possess distinct epitopes and elicit different antibody responses.

Ara h 6, a conglutin seed storage protein and member of the 2S albumin family. Ara h 6 has a molecular weight of 15 kilodaltons (kDa) and shares about 59% of its amino acid sequence identity with Ara h 2. Ara h 6 is also recognized as a potent allergen protein.

Ara h 8, with a molecular mass of 17 kilodaltons (kDa), belongs to the PR-10 protein family. Two isoforms of Ara h 8 have been distinguished: Ara h 8.0101 and Ara h 8.0201. These isoforms may exhibit distinct characteristics and play varying roles in plant immunity and allergenicity.

Ara h 9 is a non-specific lipid-transfer protein (nsLTP), identified as a peanut allergen component with a molecular mass of 9.8 kilodaltons (kDa). The primary biological role involves facilitating the transport of phospholipids and other fatty acids across cell membranes. Ara h 9 is stable and can endure thermal and protease treatment. Ara h 9 exists in two isoforms, namely Ara h 9.0101 and Ara h 9.0201, both sharing 90% sequence identity with Ara h 9.

Excipients can be added to the modified peanut allergen protein composition to improve stability and efficacy, but careful consideration may be given to the potential impact of these additives on the safety and immunogenicity of the final product. For instance, when using dialdehyde to modify Ara h 2, the excipients chosen for the dosage form may affect the overall stability and efficacy of the modified protein. The molecular weight of the modified protein can also influence its efficacy and safety for use in desensitization therapy.

The primary objectives of incorporating excipients are to maintain the reduced allergenicity and preserved T-cell reactivity of the modified proteins during manufacturing, storage, and administration.

Excipients may also be included in the modified peanut allergen protein compositions to facilitate or improve the delivery of the allergen proteins to the immune system in a manner that enhances desensitization efficacy while minimizing adverse reactions. Delivery-enhancing excipients may function by modulating the rate of allergen release, promoting targeted uptake by antigen-presenting cells, or improving tissue compatibility at the site of administration.

Suitable stabilizing excipients include, but are not limited to, disaccharides such as sucrose and trehalose, and polyols such as mannitol, sorbitol, and glycerol. These agents may stabilize the tertiary and quaternary structures of the modified allergen proteins by forming a protective matrix or by replacing water molecules, thereby preventing conformational changes, aggregation, or denaturation that could compromise the safety and efficacy of the composition.

Exemplary excipients may also include bulking agents, for volume enhancement and texture improvement, include microcrystalline cellulose, maltodextrin, polydextrose, inulin, sorbitol, and calcium carbonate. Microcrystalline cellulose, derived from cellulose, is a common choice for pharmaceutical tablets and capsules. Maltodextrin, a starch-derived polysaccharide, is favored in food products and some pharmaceuticals due to its solubility and mild flavor. Polydextrose and inulin serve as soluble fibers and bulking agents in food products and supplements. Sorbitol, a sugar alcohol, adds bulk and sweetness to sugar-free and low-calorie foods. Calcium carbonate, a mineral, acts as a bulking agent and filler in pharmaceutical formulations. These agents are selected based on compatibility, desired properties, and regulatory standards, crucial for achieving desired characteristics while maintaining quality and acceptability.

Excipients are substances that are added to a pharmaceutical formulation to provide various functional benefits such as stabilization, solubility enhancement, and improved bioavailability. In the context of the present disclosure, excipients can be incorporated into the modified peanut allergen protein composition to improve its stability, efficacy, and overall safety profile.

The choice of excipient will depend on various factors such as the desired properties of the final product, the chosen route of administration, and the specific requirements of the patient population being treated. Some common excipients used in allergen-specific immunotherapy include buffers, surfactants, and stabilizers.

Buffers are substances that help maintain a stable pH environment for the modified peanut allergen protein, which can be particularly valuable if the modified protein is being formulated as a liquid dosage form. Common buffering agents used in allergen-specific immunotherapy include acetic acid, sodium hydroxide, and phosphoric acid.

Surfactants are substances that reduce the surface tension of a solution, allowing for improved dispersion and dissolution of the modified peanut allergen protein. This can be particularly beneficial if the modified protein is poorly soluble in water or other solvents used in formulation. Common surfactants used in allergen-specific immunotherapy include polysorbate 80, lecithin, and polyoxylethylene glycol stearate.

Stabilizers are substances that help protect the modified peanut allergen protein from degradation or aggregation during storage and distribution. Common stabilizers used in allergen-specific immunotherapy include polysaccharides such as carboxymethylcellulose, hydroxypropyl methylcellulose, and gum arabic.

The addition of excipients can also help improve the bioavailability of the modified peanut allergen protein, which is useful for its efficacy in desensitization therapy. This can be achieved through the use of formulation strategies such as controlled-release formulations or liposomal encapsulation.

However, careful consideration may be given to the potential impact of excipients on the safety and immunogenicity of the final product. Some excipients may trigger allergic reactions in sensitive individuals or interfere with the efficacy of the modified peanut allergen protein. Therefore, it is useful to thoroughly evaluate the safety profile of any excipient used in the formulation process.

In addition, the choice and quantity of excipients may be carefully optimized to ensure that they do not compromise the efficacy or safety of the modified peanut allergen protein. This may require extensive testing and optimization of various formulations before a final product is selected for clinical use.

Solid and liquid dosage forms are a useful aspect of pharmaceutical formulation, allowing for efficient delivery of active ingredients like modified peanut allergen protein to patients. In the context of the present disclosure, solid and liquid dosage forms can be used to deliver the chemically modified peanut allergen protein in a controlled manner, ensuring consistent exposure to the patient and minimizing potential side effects.

The choice between solid or liquid dosage forms will depend on various factors such as the route of administration, desired release profile, stability characteristics, and patient preferences. For instance, if the modified peanut allergen protein is administered subcutaneously or intramuscularly, a liquid dosage form may be preferred to facilitate injection. In contrast, if the modified protein needs to be delivered orally, a solid dosage form like a capsule or tablet can be used for ease of administration and improved bioavailability.

The specific design of solid and liquid dosage forms containing modified peanut allergen protein will depend on several factors such as the chemical modification method employed, average molecular weight of the modified protein, and desired release characteristics. Controlled-release formulations can be used to gradually release the modified protein over time, mimicking the natural exposure pattern of the allergen in the body. This approach can help minimize potential side effects and enhance efficacy by reducing the risk of rapid onset of adverse reactions during administration.

In addition to the active ingredient, solid and liquid dosage forms may also contain excipients that are added for various purposes such as improving stability, enhancing bioavailability, or modifying release properties. These excipients should be carefully selected to ensure compatibility with the modified peanut allergen protein and minimize potential adverse effects on patient safety and efficacy.

The chemically modified peanut protein can be formulated into a variety of pharmaceutical products that can be tailored to different routes of administration, including oral, topical, subcutaneous, and intramuscular routes. For oral administration, formulations such as capsules, tablets, or oral suspensions can be designed to ensure convenient ingestion and efficient absorption in the gastrointestinal tract. These formulations are particularly suitable for managing peanut allergies through desensitization protocols or providing therapeutic relief for gastrointestinal disorders.

Topical formulations, including creams, ointments, or patches, present an effective approach for targeting localized skin conditions associated with peanut allergies or other dermatological issues. These formulations allow for direct application to the skin, minimizing systemic adverse reaction.

Subcutaneous injections enable the delivery of modified peanut protein beneath the skin layer, facilitating controlled release and systemic distribution of the medication. This route is commonly utilized in immunotherapy protocols for gradually desensitizing individuals to peanut allergens and reducing allergic reactions over time.

Similarly, intramuscular injections deposit the modified peanut protein into muscle tissue, ensuring systemic distribution and prolonged therapeutic effects. This route is often employed for the administration of vaccines or long-acting medications targeting peanut allergies or related immune disorders.

A strategic approach for reducing the allergenic potential of protein-containing allergens involves chemically modifying the allergen using a dialdehyde compound, such as glutaraldehyde or glyoxal. This process is designed to diminish IgE-mediated hypersensitivity responses while preserving the immunogenic characteristics necessary to induce immune tolerance. The modification is achieved by reacting the allergen protein with the dialdehyde under controlled conditions, resulting in covalent cross-linking of amino groups, primarily lysine side chains, and other nucleophilic residues such as free thiols on cysteine residues. These cross-links alter the protein's tertiary and quaternary structures, effectively masking conformational IgE-binding epitopes that trigger allergic reactions. However, because T-cell epitopes are often linear and less dependent on native protein conformation, the modified allergen can still engage T-cell receptors, thereby facilitating immune modulation.

The modified allergen, commonly referred to as an “allergoid,” can be formulated for administration via subcutaneous, sublingual, or other immunotherapeutic routes, optionally in combination with an adjuvant such as aluminum hydroxide or microcrystalline tyrosine.

Exemplary allergic conditions that may be treated using chemically modified allergen proteins, such as dialdehyde-treated allergoids, span a broad range of IgE-mediated hypersensitivities affecting GIT, skin, respiratory and systemic immune systems. These conditions include, but are not limited to:

Allergic rhinitis and seasonal allergic rhinoconjunctivitis, which are often triggered by airborne allergens such as grass pollen, tree pollen (e.g., birch, oak), weed pollen (e.g., ragweed), and fungal spores (e.g., Alternaria, Cladosporium). In these cases, native pollen proteins can be chemically modified with dialdehydes to produce polymerized allergoids that maintain T-cell reactivity while minimizing IgE-mediated cross-linking and mast cell activation upon administration.

Allergic asthma, particularly when associated with sensitization to environmental allergens such as house dust mites (e.g., Dermatophagoides pteronyssinus, Dermatophagoides farinae), cockroach allergens, or animal dander (e.g., cat or dog). Modified protein allergens from these sources can be incorporated into immunotherapeutic regimens aimed at reducing airway hyperresponsiveness and eosinophilic inflammation by promoting regulatory T-cell responses without provoking exacerbations.

Food allergies, including peanut, tree nut (e.g., cashew, walnut, hazelnut), egg, milk, wheat, soy, and shellfish allergies, are increasingly being explored for immunotherapeutic intervention. Although clinical immunotherapy for food allergy remains limited, research into the use of chemically modified food proteins, such as dialdehyde-crosslinked peanut allergens (e.g., Ara h 1, Ara h 2), is ongoing. The goal is to reduce the risk of anaphylaxis while establishing tolerance through controlled exposure to immunologically active but non-reactogenic protein forms.

Insect venom allergy, such as hypersensitivity to bee venom (Apis mellifera), wasp venom (Vespula spp.), or fire ant venom, also represents a major therapeutic target. Venom proteins can be chemically modified using dialdehydes to reduce systemic reactivity and permit safer dose escalation during venom immunotherapy (VIT), which remains the standard of care for preventing future anaphylactic events in sensitized individuals.

Atopic dermatitis, particularly when associated with sensitization to environmental or food allergens, may also benefit from systemic desensitization via modified allergen immunotherapy. While the relationship between allergen exposure and atopic skin disease is complex, dialdehyde-treated allergen preparations may contribute to long-term immune tolerance and reduce flare frequency in select patients.

Collectively, these conditions illustrate the wide applicability of dialdehyde-mediated chemical modification strategies in designing safer, immunologically active allergen preparations for both investigational and clinical use in allergen-specific immunotherapy.

### Oral Immunotherapy Regimen Utilizing Chemically Crosslinked Peanut Allergoids

In one embodiment, the present disclosure provides an oral immunotherapy (OIT) regimen for inducing desensitization to peanut allergens in individuals with IgE-mediated peanut allergy. The method comprises the controlled oral administration of a composition comprising one or more chemically modified peanut proteins, herein referred to as “allergoids,” which have been structurally altered by covalent crosslinking using a dialdehyde reagent, such as glutaraldehyde or glyoxal as previously described. The allergoids are formulated to reduce IgE-binding activity while retaining immunogenic T-cell epitopes, thereby enabling effective immunomodulation with reduced risk of anaphylaxis.

The OIT regimen includes an initiation phase, a dose escalation phase, and a maintenance phase, as described below.

### Initiation Phase

Prior to commencing therapy, patients may be screened to confirm eligibility based on a diagnosis of peanut allergy, determined via clinical history, skin prick testing, oral food challenge, and/or peanut-specific serum IgE levels. On the first day of treatment, an initial dose of the crosslinked peanut allergoid composition, equivalent to approximately 0.1 mg to 1 mg of total peanut protein, is administered under medical supervision in a clinical setting. The patient is observed for a period of 1 to 2 hours to monitor for hypersensitivity reactions. If no clinically significant adverse events occur, the patient may continue with once-daily administration of the same dose at home.

### Dose Escalation Phase

Following successful completion of the initiation phase, the patient enters a dose escalation phase wherein the daily dose of the crosslinked allergoid composition is increased in incremental steps. Each up-dosing step may be conducted under medical supervision at intervals of approximately 1 to 2 weeks. The dosing increments may follow a logarithmic or modified linear schedule, for example: 1 mg, 3 mg, 10 mg, 30 mg, 100 mg, 300 mg, and 600 mg of peanut protein equivalents. The specific amount and timing of each dose escalation may be adjusted based on patient tolerance and clinical judgment.

The crosslinked allergoid composition may be administered in a neutral edible medium such as an aqueous suspension, a semi-solid food matrix (e.g., yogurt, pudding, or applesauce), or encapsulated form to facilitate dosing accuracy and palatability.

### Maintenance Phase

Upon achieving the target maintenance dose: typically between 300 mg and 600 mg of peanut protein equivalents per day, the patient transitions to a maintenance phase. In this phase, the same dose is administered orally once per day to once weekly for a duration of at least 6 months, and preferably 12 months or longer. Continued administration is intended to reinforce and sustain the desensitization response.

Periodic clinical evaluations may be performed to assess treatment adherence, symptom profile, and immunologic changes such as increases in peanut-specific IgG4 levels, decreases in IgE levels, and reductions in skin test reactivity. In some embodiments, an optional double-blind placebo-controlled food challenge (DBPCFC) may be conducted after 12 to 18 months of maintenance therapy to evaluate the subject's threshold of clinical reactivity to unmodified peanut protein.

The described OIT regimen may be adapted for pediatric or adult subjects and may be co-administered with adjunctive agents such as antihistamines, leukotriene receptor antagonists, or biologics (e.g., anti-IgE antibodies) to further enhance safety and efficacy.

Incorporating chemically crosslinked allergoids into an OIT regimen represents a novel strategy for reducing the risk of systemic allergic reactions while facilitating robust immune desensitization to peanut allergens.

### Subcutaneous Immunotherapy Regimen Utilizing Chemically Crosslinked Peanut Allergoids

In another embodiment, the present disclosure provides a method of desensitizing a subject to peanut allergens by administering a composition comprising chemically crosslinked peanut proteins via subcutaneous injection. The peanut proteins are chemically modified through crosslinking with a bifunctional aldehyde compound, such as glutaraldehyde or glyoxal, to generate allergoids with reduced IgE-binding capacity while retaining immunologically active epitopes necessary for T-cell mediated immune modulation.

The subcutaneous immunotherapy (SCIT) regimen includes three phases: an initial dose escalation (induction), a build-up phase, and a maintenance phase.

### Composition

The immunotherapeutic composition comprises one or more modified peanut allergenic proteins, such as Ara h 1, Ara h 2, and Ara h 6, which have been treated with a dialdehyde to induce intermolecular crosslinking and structural stabilization. The resultant allergoids exhibit significantly reduced capacity to crosslink IgE antibodies on mast cells or basophils, thereby mitigating the risk of systemic hypersensitivity reactions. The modified allergens may be formulated with a depot adjuvant such as aluminum hydroxide, microcrystalline tyrosine (MCT), or monophosphoryl lipid A (MPL) to enhance and prolong antigen presentation and facilitate tolerogenic immune responses.

### Induction and Build-Up Phase

The method comprises administering subcutaneous injections of the allergoid composition in gradually increasing doses. An initial induction dose may range from approximately 0.01 μg to 1 μg of peanut protein equivalent, delivered as a single injection under clinical supervision. Subsequent doses are escalated according to a standardized schedule over the course of several weeks or months, typically involving weekly or biweekly injections.

A representative escalation protocol may include dose levels of 1 μg, 5 μg, 20 μg, 50 μg, 100 μg, 200 μg, and 300 μg peanut protein equivalents. Each injection is administered subcutaneously into the upper arm or another appropriate site, and the subject is monitored for adverse reactions for at least 30 minutes post-injection.

The build-up phase is designed to achieve a target maintenance dose that is sufficient to induce immune tolerance without provoking significant IgE-mediated symptoms.

### Maintenance Phase

Upon reaching the target dose, typically 300 μg to 500 μg of peanut protein equivalent, the subject transitions to the maintenance phase. During this phase, the subject receives subcutaneous injections of the allergoid composition at fixed intervals, typically once every 2 to 4 weeks, for a period of 12 months or more. The maintenance regimen is intended to promote durable desensitization and may be continued indefinitely based on clinical judgment and immunologic markers.

Periodic evaluations may be conducted to monitor serum peanut-specific IgE and IgG4 levels, T-cell cytokine profiles, and symptom response to incidental peanut exposure. In certain embodiments, a double-blind placebo-controlled food challenge (DBPCFC) or open oral food challenge may be used to confirm desensitization.

### Intramuscular Immunotherapy Regimen Utilizing Chemically Crosslinked Peanut Allergoids

In another embodiment, the present disclosure provides a method of desensitizing a subject to peanut allergens by administering a composition comprising chemically crosslinked peanut proteins via intramuscular injection. The peanut proteins are chemically modified through crosslinking with a bifunctional aldehyde compound, such as glutaraldehyde or glyoxal, to generate allergoids with reduced IgE-binding capacity while retaining immunologically active epitopes necessary for T-cell mediated immune modulation.

The intramuscular immunotherapy (IMIT) regimen includes three phases: an initial dose escalation (induction), a build-up phase, and a maintenance phase.

### Composition

The immunotherapeutic composition comprises one or more modified peanut allergenic proteins, such as Ara h 1, Ara h 2, and Ara h 6, that have been crosslinked using a dialdehyde agent to induce intermolecular bonding and structural stabilization. The resulting allergoids exhibit reduced capacity to crosslink IgE on mast cells or basophils, thereby lowering the potential for systemic allergic reactions. The modified proteins may be formulated with a depot-forming or immunomodulatory adjuvant such as aluminum hydroxide, microcrystalline tyrosine (MCT), or monophosphoryl lipid A (MPL), to enhance antigen retention at the injection site and prolong immune system engagement.

### Induction and Build-Up Phase

The IMIT method comprises administering intramuscular injections of the allergoid composition in gradually increasing doses. An initial induction dose may range from approximately 0.01 μg to 1 μg of peanut protein equivalent, delivered as a single injection into a large muscle group (e.g., the deltoid or gluteus muscle) under clinical supervision. Subsequent doses are escalated according to a predefined schedule over several weeks or months, typically involving injections every 2 to 4 weeks.

A representative escalation protocol may include dose levels of 1 μg, 5 μg, 20 μg, 50 μg, 100 μg, 200 μg, and 300 μg peanut protein equivalents. Each dose is administered intramuscularly, and the subject is observed for adverse reactions for at least 30 minutes post-injection. The build-up phase is designed to gradually achieve a target maintenance dose sufficient to promote immune tolerance without triggering clinically significant allergic responses.

### Maintenance Phase

Upon reaching the target dose, typically between 300 μg and 500 μg of peanut protein equivalent, the subject transitions to a maintenance phase. During this phase, the subject receives intramuscular injections of the allergoid composition at fixed intervals, generally every 3 to 6 weeks, for a period of 12 months or longer. The maintenance regimen is intended to sustain immune desensitization over time and may be continued as needed based on clinical evaluation.

Periodic assessments may be conducted to monitor serum peanut-specific IgE and IgG4 levels, T-cell cytokine profiles, and clinical response to incidental peanut exposure. In certain embodiments, desensitization may be confirmed using a double-blind placebo-controlled food challenge (DBPCFC) or open oral food challenge conducted under medical supervision.

### Epicutaneous Immunotherapy Regimen Utilizing Chemically Crosslinked Peanut Allergoids

Epicutaneous Immunotherapy (EPIT) is an allergen-specific immunotherapy method wherein an allergen is delivered across the skin barrier to induce immune tolerance while minimizing systemic allergen exposure. In EPIT, a defined dose of allergen is applied to intact skin, typically using an adhesive patch device designed to create a controlled microenvironment that facilitates allergen uptake. The allergen diffuses into the outer layers of the epidermis where it is captured by skin-resident antigen-presenting cells, such as Langerhans cells and dermal dendritic cells, which then traffic to draining lymph nodes to prime regulatory immune responses.

The epicutaneous route promotes the engagement of the immune system under conditions that favor the induction of regulatory T cells (Tregs), modulation of allergen-specific antibody production, and reduction of Th2-driven allergic inflammation. Importantly, EPIT enables immunomodulation while avoiding direct entry of the allergen into the bloodstream, thereby lowering the risk of systemic allergic reactions such as anaphylaxis.

One example of an EPIT system is the Viaskin® patch (developed by DBV Technologies), which is designed to deliver peanut protein to the skin surface for the treatment of peanut allergy. The Viaskin® system uses an electrostatically charged film to deposit a dry powder allergen onto the skin, and relies on transepidermal water loss to create a hydrated environment under the patch, facilitating allergen solubilization and absorption into the skin. Clinical studies have demonstrated that EPIT with the Viaskin® Peanut patch can safely increase the threshold of peanut reactivity in allergic individuals, particularly in pediatric populations, with a favorable safety profile characterized primarily by mild to moderate local skin reactions.

In certain embodiments, the modified peanut allergen protein compositions described herein, comprising chemically crosslinked peanut allergoids, may be formulated for administration via epicutaneous immunotherapy (EPIT). The composition may be incorporated into a transdermal delivery system, such as an adhesive patch configured to deliver the modified peanut proteins to the epidermis in a controlled manner. The patch may optionally comprise a hydrated chamber or moisture-retentive membrane to facilitate solubilization of the modified proteins upon application to the skin surface. The chemically crosslinked peanut allergoids may provide the advantages of reduced IgE binding and enhanced stability while preserving T-cell epitopes necessary for the induction of desensitization or immune tolerance. Epicutaneous administration of the modified allergen compositions may permit gradual exposure of the immune system to the allergens, thereby reducing the risk of systemic allergic reactions and improving the safety profile of the desensitization therapy.

In certain embodiments, the allergen may be formulated with stabilizers, wetting agents, or skin-permeation enhancers to improve the efficacy of epicutaneous delivery. The patch device may be configured to maintain the allergen in a hydrated or semi-hydrated state, which promotes consistent and gradual uptake through the stratum corneum.

The term “peanut protein equivalent” as used herein refers to the amount of peanut-derived protein present in a composition, expressed in terms of the mass of native, unmodified peanut protein from which the composition is derived. The term is used to standardize dosing across different forms of allergen preparations, including but not limited to chemically modified, purified, or crosslinked allergenic proteins, such as allergoids.

For example, where a peanut protein such as Ara h 1, Ara h 2, or Ara h 6 has been chemically modified by crosslinking with a dialdehyde reagent (e.g., glutaraldehyde or glyoxal), the resulting allergoid may be structurally and immunologically distinct from the native protein. Nevertheless, the composition may be characterized by the mass of native peanut protein that was initially present prior to chemical modification. Accordingly, a dose comprising 1 mg of crosslinked peanut protein that was derived from 1 mg of native peanut protein is considered to contain 1 mg of “peanut protein equivalent.”

This standardization enables consistent comparison of immunotherapeutic dosages and facilitates regulatory, clinical, and manufacturing alignment. Quantification of peanut protein equivalents may be carried out using total protein assays (e.g., bicinchoninic acid assay, Lowry assay), nitrogen content analysis, or immunoreactivity-based methods such as ELISA directed against linear or conformational epitopes of native peanut allergens.

All documents mentioned in this specification are incorporated herein by reference in their entirety for all purposes.

What is claimed:

1. A composition of peanut allergen protein allergoid, comprising at least one peanut allergen protein selected from the group consisting of: Ara h 1, 2, 3, 6, 8, and 9,
wherein the peanut allergen protein is chemically modified by crosslinking with a dialdehyde, and
wherein the chemically modified peanut allergen protein demonstrated reduced binding affinity with IgE.

2. The composition of claim 1, wherein the chemical modification is by crosslinking with a glyoxal.

3. The composition of claim 1, wherein the chemical modification is by crosslinking with a phenyl glyoxal.

4. The composition of claim 3, wherein the chemical modification is by crosslinking with at least one of phenylglyoxal, 4-methylphenylglyoxal, 4-methoxyphenylglyoxal, 4-fluoro-phenylglyoxal, 2,4-difluorophenylglyoxal, 3,4-difluorophenylglyoxal, 4-chlorophenylglyoxal,4-hydroxyphenylglyoxal, 3-carbomethoxy-4-hydroxyphenylglyoxal, 4-morpholino-phenylglyoxal, and 4-nitrophenylglyoxal.

5. The composition of claim 3, wherein the chemical modification is by crosslinking with phenylglyoxal or 4-fluorophenylglyoxal.

6. The composition of claim 1, wherein the chemically modified peanut allergen protein having an average molecular weight greater than an average molecular wight of a native peanut allergen protein.

7. The composition of claim 6, wherein the chemically modified peanut allergen protein having an average molecular weight about 1-2 fold of an average molecular wight of a native peanut allergen protein.

8. The composition of claim 6, wherein the chemically modified peanut allergen protein having an average molecular weight about 2-fold of an average molecular wight of a native peanut allergen protein.

9. The composition of claim 1, wherein the chemically modified peanut allergen protein is at least one of Ara h 1, 2, and 6.

10. The composition of claim 9, wherein the chemically modified peanut allergen protein is Ara h 2 modified with phenylglyoxal or 4-fluorophenylglyoxal.

11. The composition of claim 1, wherein the chemical modification is by crosslinking at a non-reducing condition.

12. The composition of claim 1, further comprising an excipient.

13. A method of treating peanut allergy in a subject in need thereof, comprising:
administering to the subject a composition comprising a chemically crosslinked peanut allergoid,
wherein the peanut allergoid comprises at least one peanut protein selected from Ara h 1, 2, 3, 6, 8, and 9,
wherein the peanut protein is chemically crosslinked using a dialdehyde crosslinking agent, and
wherein the composition is administered in an amount effective to induce desensitization or immune tolerance to peanut allergens.

14. The method of claim 13, wherein the dialdehyde crosslinking agent comprises a glyoxal.

15. The method of claim 13, wherein the dialdehyde crosslinking agent comprises a phenyl glyoxal.

16. The method of claim 13, wherein the chemically modified peanut allergen protein is at least one of Ara h 1, 2, and 6.

17. The method of claim 16, wherein the chemically modified peanut allergen protein is Ara h 2 modified with phenylglyoxal or 4-fluorophenylglyoxal.

18. The method of claim 13, wherein the composition is administered according to a dose-escalation protocol comprising an initiation phase, a build-up phase, and a maintenance phase.

19. The method of claim 13, wherein the composition is administered orally at an interval of once daily to once weekly.

20. The method of claim 13, wherein the composition is administered subcutaneously at an interval of one to four weeks.

21. The method of claim 13, wherein the composition is administered intramuscularly at an interval of one to four weeks.

22. The method of claim 13, wherein the composition is administered epicutaneously.

23. The method of claim 13, wherein the subject is initially administered a dose of less than 5 mg of peanut protein equivalent and the dose is incrementally increased to a maintenance dose of at least 300 mg of peanut protein equivalent.

24. The method of claim 13, wherein the chemically crosslinked peanut allergoid has reduced IgE-binding capacity relative to the corresponding unmodified peanut protein.
